(19) United States
(12) Patent Application Publication
(10) Pub. No.: US 2025/0346905 A1
(43) Pub. Date: Nov. 13, 2025

(54) INTERFERING RNA THERAPY FOR PLN-R14del CARDIOMYOPATHY

(71) Applicants: The Board of Trustees of the Leland Stanford Junior University, Stanford, CA (US); Stichting Genetische Hartspierziekte PLN, Wieringerwerf (NL)

(72) Inventors: Ioannis Karakikes, Stanford, CA (US); Pieter Glijnis, Middenmeer (NL)

(73) Assignees: The Board of Trustees of the Leland Stanford Junior University, Stanford, CA (US); Stichting Genetische Hartspierziekte PLN, Wieringerwerf (NL)

(21) Appl. No.: 18/859,562

(22) PCT Filed: May 4, 2023
(86) PCT No.: PCT/US2023/021008

§ 371 Date: Oct. 24, 2024

Related U.S. Application Data
(60) Provisional application No. 63/338,749, filed on May 5, 2022.

(51) Int. Cl. C12N 15/113

(52) U.S. Cl. CPC C12N 15/1138; C12N 2310/14; C12N 2320/34

(57) ABSTRACT

Phospholamban (PLN) is a critical regulator of calcium cyclin and contractility in the heart. The deletion of Arginine 14 of the phospholamban gene (R14del) is associated with the pathogenesis of an inherited form of cardiomyopathy with prominent arrhythmias. Although the genetic etiology is well defined, there are currently no therapies for this rare disease. This disclosure provides an allele-specific silencing approach by interfering RNA (RNAi) to reduce the expression levels of the R14del allele of the PLN gene.

### CROSS-REFERENCE TO RELATED APPLICATIONS

This application claims priority to U.S. Provisional Application No. 63/338,749, filed May 5, 2022, the disclosure of which is herein incorporated by reference in its entirety for all purposes.

### STATEMENT AS TO RIGHTS TO INVENTIONS MADE UNDER FEDERALLY SPONSORED RESEARCH AND DEVELOPMENT

This invention was made with Government support under grant numbers R01 HL150414 and R01 HL139679 awarded by the National Institutes of Health. The Government has certain rights in the invention.

### BACKGROUND

Dilated cardiomyopathy is a form of heart disease where the heart is unable to pump blood as efficiently due to thinning (dilation) of the ventricles. Patients exhibit numerous symptoms of progressive heart failure, including ventricular arrhythmias, swelling of legs and feet, breathlessness, coughing while laying down, and fatigue. There is presently no cure for this disease, and the median survival time of a person diagnosed with dilated cardiomyopathy is roughly five years.

Several genetic mutations are associated with the development of dilated cardiomyopathy. One of these is the R14del mutation in the phospholamban gene, an allele that has been found in patients throughout the world, including the USA, Canada, China, Germany, Spain, and the Netherlands. In the Netherlands, this mutation appears to be particularly prevalent due to founder effects, and it is estimated that the Dutch population has thousands of R14del allele carriers. Among patients diagnosed with cardiomyopathy, carriers of the R14del allele suffer a particularly high incidence of malignant arrhythmias, sudden cardiac death, and cardiac transplantation. No homozygous carriers of the R14del allele have been identified.

Mouse models of R14del-mediated cardiomyopathy displayed similar phenotypes to human R14del cardiomyopathy, yet the established heart failure medications eplerenone and metoprolol were unable to improve cardiac function or survival in these models (Eijenraam et al., Scientific Reports, 10:9819 (2020)). Therefore, there is a need for therapies for patients diagnosed with dilated cardiomyopathy, particularly within the population of R14del allele carriers.

Phospholamban is a pentameric integral membrane protein encoded by the PLN gene that regulates Ca²⁺ cycling in cardiac muscle cells, an activity important for cardiac relaxation and contraction. It does so by regulating the activity of the cardiac isoform of the sarcoplasmic reticulum Ca²⁺ ATPase (SERCA2a). Ca²⁺ cycling is important for contractility (the strength and vigor of heart muscle contraction, often measured by the volume of liquid moved in a single heart contraction). Superinhibition of SERCA2a reduces contractility.

Studies in mice have suggested that R14del variants of phospholamban act synergistically with normal phospholamban to superinhibit SERCA2a, potentially by altering the structure of protein complex (Haghighi et al., PNAS, 103:1388-93 (2006)). Excessive phospholamban activity is correlated with heart failure. Furthermore, ablation of phospholamban was initially reported to have few negative consequences (Luo et al., Circulation Research, 75:401-9 (1994)). Hence, some recent studies have focused on improving cardiac function by inhibiting wild-type phospholamban using antisense oligonucleotides (ASOs) or RNAi (see, e.g., Beverborg et al., Nature Communications, 12:5180 (2021) and Suckau et al., Circulation, 119(9):1241-52 (2009)).

Genetic evidence suggests that reducing PLN expression by 50% is tolerated in humans. A mutation that converts the codon for Leu39 to the stop codon TGA has no effect in heterozygous carriers but results in dilated cardiomyopathy and premature death in the homozygous state. See Haghighi et al., J Clin Invest. 11:869-76 (2003). Similarly, a nonsense variant in PLN (p.Glu2Ter. c.4G>T) that is tolerated in heterozygous individuals is detrimental in homozygous patients. See Li et. al., Int J Cardiol. 279:122-25 (2019).

Other recent studies suggest that cardiac function in R14del carriers can be improved by permanently altering the R14del allele using transcription activator-like effector nucleases (TALENs) (see, e.g., Karakikes et al., Nature Communications, 6:6955 (2014)). However, there are drawbacks to permanently altering a genome, particularly in light of the potential for off-target mutations. Genome editing requires generation of double strand breaks (DSBs). DSB-mediated repair mechanisms, non-homologous end joining (NHEJ), and homology-directed repair (HDR) mechanisms often result in permanent undesired outcomes, including deletions, insertions, and translocations on-and off-target. These undesired outcomes have been a significant concern for translating therapeutic gene editing to the clinic. Furthermore, although DSB-mediated HDR allows the incorporation of exogenous donor templates for precise genome editing, HDR is highly inefficient in terminally differentiated, post-mitotic cells, such as cardiomyocytes. These are major roadblocks in therapeutic applications of therapeutic genome editing, especially for the many cardiovascular diseases, such as genetic cardiomyopathies, diseases involving mutations that affect post-mitotic cardiomyocytes. Therefore, there is a need for new therapies that efficiently target the R14del allele in carriers and restore normal cardiac activity without the drawbacks of gene therapy. The present disclosure satisfies this need, and provides related advantages as well.

### BRIEF SUMMARY

In some aspects, the present disclosure provides an interfering RNA molecule comprising a double-stranded region of about 15 to about 60 nucleotides in length, wherein the double-stranded region comprises a first nucleic acid strand and a second nucleic acid strand, wherein the interfering RNA molecule is capable of inhibiting expression of the R14del allele but not the wild-type allele of the phospholamban (PLN) gene.

In certain embodiments, the interfering RNA molecule comprises an siRNA molecule, an miRNA molecule, or a combination thereof.

In some embodiments, the second nucleic acid strand is substantially complementary to the first nucleic acid strand. In other embodiments, the second nucleic acid strand is fully complementary to the first nucleic acid strand. In some embodiments, the double-stranded region contains mismatches that promote duplex unwinding.

In some embodiments, the double-stranded region is 18 to 24 nucleotides in length. In some embodiments, the double-stranded region is about 20 to about 30 nucleotides in length. In some embodiments, the double-stranded region is about 25 nucleotides in length.

In some embodiments, the first nucleic acid strand has at least 90% identity to SEQ ID NO: 3. In some embodiments, the first nucleic acid strand comprises SEQ ID NO: 3. In some embodiments, the second nucleic acid strand has at least 90% identity to SEQ ID NO: 4. In some embodiments, the second nucleic acid strand comprises SEQ ID NO: 4. In some embodiments, the double-stranded region of the interfering RNA molecule comprises a first nucleic acid strand comprising at least 90% identity to SEQ ID NO: 3 and/or a second nucleic acid strand comprising at least 90% identity to SEQ ID NO: 4. In a particular embodiment, the double-stranded region of the interfering RNA molecule comprises a first nucleic acid strand comprising SEQ ID NO: 3 and a second nucleic acid strand comprising SEQ ID NO: 4.

In some embodiments, wherein the first nucleic acid strand has at least 90% identity to SEQ ID NO: 5. In some embodiments, the first nucleic acid strand comprises SEQ ID NO: 5. In some embodiments, wherein the first nucleic acid strand has at least 90% identity to SEQ ID NO: 6. In some embodiments, the first nucleic acid strand comprises SEQ ID NO: 6. In some embodiments, the double-stranded region of the interfering RNA molecule comprises a first nucleic acid strand comprising at least 90% identity to SEQ ID NO: 5 and/or a second nucleic acid strand comprising at least 90% identity to SEQ ID NO: 6. In a particular embodiment, the double-stranded region of the interfering RNA molecule comprises a first nucleic acid strand comprising SEQ ID NO: 5 and a second nucleic acid strand comprising SEQ ID NO: 6.

In some embodiments, wherein the first nucleic acid strand has at least 90% identity to SEQ ID NO: 7. In some embodiments, the first nucleic acid strand comprises SEQ ID NO: 7. In some embodiments, wherein the first nucleic acid strand has at least 90% identity to SEQ ID NO: 8. In some embodiments, the first nucleic acid strand comprises SEQ ID NO: 8. In some embodiments, the double-stranded region of the interfering RNA molecule comprises a first nucleic acid strand comprising at least 90% identity to SEQ ID NO: 7 and/or a second nucleic acid strand comprising at least 90% identity to SEQ ID NO: 8. In a particular embodiment, the double-stranded region of the interfering RNA molecule comprises a first nucleic acid strand comprising SEQ ID NO: 7 and a second nucleic acid strand comprising SEQ ID NO: 8.

In some embodiments, wherein the first nucleic acid strand has at least 90% identity to SEQ ID NO: 9. In some embodiments, the first nucleic acid strand comprises SEQ ID NO: 9. In some embodiments, wherein the first nucleic acid strand has at least 90% identity to SEQ ID NO: 10. In some embodiments, the first nucleic acid strand comprises SEQ ID NO: 10. In some embodiments, the double-stranded region of the interfering RNA molecule comprises a first nucleic acid strand comprising at least 90% identity to SEQ ID NO: 9 and/or a second nucleic acid strand comprising at least 90% identity to SEQ ID NO: 10. In a particular embodiment, the double-stranded region of the interfering RNA molecule comprises a first nucleic acid strand comprising SEQ ID NO: 9 and a second nucleic acid strand comprising SEQ ID NO: 10.

In some aspects, the interfering RNA molecule comprises a 3′ overhang in the first nucleic acid strand and/or the second nucleic acid strand. In some embodiments, one or more of the nucleotides in the double-stranded region comprise modified nucleotides.

In some embodiments, the interfering RNA molecule further comprises a carrier system.

The present disclosure also provides a pharmaceutical composition comprising an interfering RNA molecule described herein and a pharmaceutically acceptable carrier.

The present disclosure further provides the disclosed herein interfering RNA molecule according to any one of the described herein embodiments, and/or the disclosed herein pharmaceutical composition comprising said interfering RNA molecule, for use as a medicament.

In advantageous embodiments, the disclosure provides the disclosed herein interfering RNA molecule according to any one of the described herein embodiments, and/or the disclosed herein pharmaceutical composition comprising said interfering RNA molecule, for use in the treatment of cardiomyopathy, preferably in a subject carrying the R14del allele of the PLN gene.

In further embodiments, the disclosure provides the disclosed herein interfering RNA molecule according to any one of the described herein embodiments, and/or the disclosed herein pharmaceutical composition comprising said interfering RNA molecule, for use in lowering the likelihood of at least one of malignant arrhythmias, sudden cardiac death, and/or a heart transplant in a subject suffering from cardiomyopathy, preferably in a subject carrying the R14del allele of the PLN gene.

In some embodiments, the disclosed herein interfering RNA molecule according to any one of the described herein embodiments, and/or the disclosed herein pharmaceutical composition comprising said interfering RNA molecule is provided for the uses as described herein, wherein preferably the treatment comprises administering to the subject a therapeutically effective amount of an interfering RNA molecule described herein or a pharmaceutical composition described herein and/or wherein the mode of administration is selected from the group consisting of oral, intranasal, intravenous, intraperitoneal, intramuscular, intraarticular, intralesional, subcutaneous, and intradermal. In some embodiments, the subject has been diagnosed with cardiomyopathy. In some embodiments, the subject is a human.

In some embodiments, disclosed herein is a use of the disclosed herein interfering RNA molecule according to any one of the described herein embodiments, for the manufacture of a medicament, in particular for the treatment of cardiomyopathy, preferably in a subject carrying the R14del allele of the PLN gene.

The present disclosure also provides a method for introducing an interfering RNA molecule that is capable of inhibiting expression of the R14del allele of the PLN gene in a cell, the method comprising contacting the cell with an interfering RNA molecule described herein or a pharmaceutical composition described herein. In some embodiments, the cell is a cardiac muscle cell. In some instances, the cell is a cardiomyocyte. In some embodiments, the cell is in a subject. In some embodiments, the subject is a carrier of the R14del allele of the PLN gene. In some embodiments, the subject has been diagnosed with cardiomyopathy. In some embodiments, the subject is a human. In some instances, the interfering RNA molecule does not affect the contractility and/or the action potential of the cardiac muscle cell. In some instances, the siRNA molecule has a positive effect on the contractility and/or the action potential of the cardiac muscle cell.

The present disclosure also provides a method for in vivo delivery of an interfering RNA molecule that silences the expression of the R14del allele of the PLN gene in a cell of a subject, the method comprising administering to the subject an interfering RNA molecule described herein, or a pharmaceutical composition described herein. In some embodiments, the cell is a cardiac muscle cell. In some embodiments, the subject is a carrier of the R14del allele of the PLN gene. In some embodiments, the subject has been diagnosed with cardiomyopathy. In some embodiments, the subject is a human.

The present disclosure also provides a method for treating cardiomyopathy in a subject carrying the R14del allele of the PLN gene, the method comprising administering to the subject a therapeutically effective amount of an interfering RNA molecule described herein or a pharmaceutical composition described herein.

The present disclosure also provides a method for lowering the likelihood of at least one of malignant arrhythmias, sudden cardiac death, and/or a heart transplant in a subject carrying the R14del allele of the PLN gene, the method comprising administering to the subject a therapeutically effective amount of an interfering RNA molecule described herein, or a pharmaceutical composition described herein.

In some embodiments, the mode of administration is selected from the group consisting of oral, intranasal, intravenous, intraperitoneal, intramuscular, intraarticular, intralesional, subcutaneous, and intradermal. In some embodiments, the subject has been diagnosed with cardiomyopathy. In some embodiments, the subject is a human.

### BRIEF DESCRIPTION OF THE DRAWINGS

FIG. 1 illustrates the overall allele-specific siRNA-based approach.

FIG. 2 illustrates siRNA-mediated knockdown of the R14del allele in iPSC cardiomyocytes (iPSC-CMs) measured by real time quantitative PCR using siRNAs designed to target R14del allele transcripts. Shown are PLN transcript levels in iPSC-CMs derived from R14del carriers (top panels) and wild-type (WT) PLN carriers (bottom panels).

FIG. 3 illustrates the ratios of WT and R14del allele transcripts following knockdown with siRNA 1, 2, or 3 or non-targeting control siRNA (siRNA neg) in two different R14del carrier-derived iPSC-CM cell lines.

FIG. 4A illustrates the difference in normalized counts (counts per million reads mapped) based on RNAseq analyses of R14del between iPSC-CMs treated with siRNA 3 (siR14.3) compared to a non-targeting control siRNA (siCtrl). FIG. 4B illustrates the reproducibility of the difference in allelic ratio of wild-type PLN to R14del PLN between iPSC-CMs treated with siRNA 3 (si14.3) compared to a non-targeting control siRNA (siCtrl).

FIG. 5 illustrates that there is no adverse effect of siRNAs on contractility when iPSC-CMs are treated with either siRNA 1, siRNA 2, or siRNA 3.

FIG. 6 illustrates that there are no adverse effects on action potential when iPSC-CMs are treated with either siRNA 1, siRNA 2, or siRNA 3.

FIG. 7 illustrates that treatment with siRNA 3 restores contractility in engineered heart tissues (EHTs) generated from iPSC-derived cardiomyocytes (iPSC-CMs) with R14del PLN.

### DETAILED DESCRIPTION

### I. INTRODUCTION

Cardiac muscle forms the walls of the heart and enables the heart to pump blood to the circulatory system. Within cardiac muscles, bundles of cardiomyocytes shorten and lengthen their myofibers, creating the pumping force in the heart. Modified cardiomyocytes called cardiac pacemaker cells create rhythmic impulses controlling the heart rate. Cardiac muscle contraction is initiated by an action potential (electrical impulse) propagated from pacemaker cells and leads to depolarization of the muscle cell plasma membrane, causing opening of calcium channels and entry of Ca²⁺ into the cells as well as the release of Ca²⁺ from the internal stores of the sarcoplasmic reticulum organelle. The resulting free Ca²⁺ causes regulatory proteins to be released from muscle motor proteins, and movement of the freed muscle motor proteins leads to muscle contraction.

Before the heart can contract again, free Ca²⁺ must be transported back to the sarcoplasmic reticulum for storage via SERCA2a (sarcoplasmic/endoplasmic reticulum Ca²⁺-ATPase), a pump in the sarcoplasmic reticulum membrane. SERCA2a activity is regulated by phospholamban, an integral membrane protein encoded by PLN. Inhibition of SERCA2a by phospholamban ultimately prevents contraction. Studies in mice have suggested that the R14del allele of PLN (encoding a mutant phospholamban protein where the fourteenth amino acid, arginine, is deleted) causes superinhibition of SERCA2a. This may explain why carriers of this allele are at high risk of developing dilated cardiomyopathy or arrhythmogenic right ventricular cardiomyopathy.

Commonly used heart failure drugs have been shown to be ineffective in mouse models of R14del-associated cardiomyopathy, and recent therapeutic approaches that have focused on abolishing the wild-type PLN gene or correcting the R14del mutation are met with significant drawbacks. The present disclosure describes a novel method of treating R14del carriers, using specific interfering RNA (e.g., siRNA) molecules that can target R14del allele transcripts, and significantly increase the ratio of wild-type to R14del PLN transcripts. Furthermore, these interfering RNAs (e.g., siRNAs) are affective at increasing the ratio of wild-type PLN in iPSC-CMs cells without negatively affecting either contractility or action potential generation.

### II. DEFINITIONS

As used herein, the following terms have the meanings ascribed to them unless specified otherwise.

The term “nucleic acid” as used herein refers to a polymer containing at least two deoxyribonucleotides or ribonucleotides in either single-or double-stranded form and includes DNA and RNA. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2′-O-methyl ribonucleotides, and peptide-nucleic acids (PNAs). Unless specifically limited, the term encompasses nucleic acids containing known analogues of natural nucleotides that have similar binding properties as the reference nucleic acid.

“Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkyl halides (haloalkanes).

“Interfering RNA,” “RNAi” or “interfering RNA sequence” as used herein refers to an RNA molecule capable of reducing or inhibiting the expression of a target gene or sequence (e.g., by mediating the degradation or inhibiting the translation of mRNAs which are complementary to the interfering RNA sequence) when the interfering RNA is in the same cell as the target gene or sequence.

Interfering RNA can be about 15-60, 15-50, or 15-40 (duplex) nucleotides in length, more typically about 15-30, 15-25, or 19-25 (duplex) nucleotides in length, and is preferably about 20-24, 21-22, or 21-23 (duplex) nucleotides in length (e.g., each complementary sequence of the double-stranded siRNA is 15-60, 15-50, 15-40, 15-30, 15-25, or 19-25 nucleotides in length, preferably about 20-24, 21-22, or 21-23 nucleotides in length, and the double-stranded siRNA is about 15-60, 15-50, 15-40, 15-30, 15-25, or 19-25 base pairs in length, preferably about 18-22, 19-20, or 19-21 base pairs in length). Interfering RNA duplexes may comprise 3′ overhangs of about 1 to about 4 nucleotides or about 2 to about 3 nucleotides and 5′ phosphate termini. Examples of interfering RNA include, without limitation, a double-stranded polynucleotide molecule assembled from two separate stranded molecules, wherein one strand is the sense strand and the other is the complementary antisense strand; a double-stranded polynucleotide molecule assembled from a single stranded molecule, where the sense and antisense regions are linked by a nucleic acid-based or non-nucleic acid-based linker; a double-stranded polynucleotide molecule with a hairpin secondary structure having self-complementary sense and antisense regions; and a circular single-stranded polynucleotide molecule with two or more loop structures and a stem having self-complementary sense and antisense regions, where the circular polynucleotide can be processed in vivo or in vitro to generate an active double-stranded interfering RNA molecule.

Interfering RNA can be chemically synthesized or generated by cleavage of longer dsRNA (e.g., dsRNA greater than about 25 nucleotides in length) with the E. coli RNase III or Dicer. These enzymes process the dsRNA into biologically active interfering RNA (see, e.g., Yang et al., Proc. Natl. Acad. Sci. USA, 99:9942-9947 (2002); Calegari et al., Proc. Natl. Acad. Sci. USA, 99:14236 (2002); Byrom et al., Ambion TechNotes, 10(1):4-6 (2003); Kawasaki et al., Nucleic Acids Res., 31:981-987 (2003); Knight et al., Science, 293:2269-2271 (2001); and Robertson et al., J. Biol. Chem., 243:82 (1968)). Preferably, dsRNA are at least 50 nucleotides to about 100, 200, 300, 400, or 500 nucleotides in length. A dsRNA may be as long as 1000, 1500, 2000, 5000 nucleotides in length, or longer. The dsRNA can encode for an entire gene transcript or a partial gene transcript. In certain instances, interfering RNA may be encoded by a plasmid (e.g., transcribed as sequences that automatically fold into duplexes with hairpin loops).

Each of the interfering RNA sequences present in the compositions of the invention may independently comprise at least one, two, three, four, five, six, seven, eight, nine, ten, or more “modified nucleotides” such as 2′-O-methyl ribonucleotides, e.g., in the sense and/or antisense strand of the double-stranded region. Such modifications can render the polynucleotide resistant to nucleases, improve delivery of the polynucleotide to target cells or tissues, improve stability, reduce degradation, improve tissue distribution or to impart other advantageous properties. For example, the DNA or RNA polynucleotide may include one or more modifications on the oligonucleotide backbone (e.g., a phosphorothioate modification), the sugar (e.g., a locked sugar), or the nucleobase. If present, modifications to the nucleotide structure can be imparted before or after assembly of the oligonucleotide. Furthermore, in order to improve the oligonucleotide delivery, the DNA or RNA oligonucleotide may be packaged into a carrier system (elaborated below) or be conjugated to a cell-penetrating peptide.

In some embodiments, each of the interfering RNA sequences described herein may independently comprise a 3′ overhang of 1, 2, 3, or 4 nucleotides in one or both strands of the interfering RNA or may comprise at least one blunt end. In certain instances, the 3′ overhangs in one or both strands of the interfering RNA each independently comprise 1, 2, 3, or 4 of any combination of modified and unmodified deoxythymidine (dT) nucleotides, 1, 2, 3, or 4 of any combination of modified and unmodified uridine (U) ribonucleotides, or 1, 2, 3, or 4 of any combination of modified and unmodified ribonucleotides having complementarity to the target sequence (3′ overhang in the antisense strand) or the complementary strand thereof (3′ overhang in the sense strand).

A “antisense strand” refers to the strand of an interfering RNA (e.g., siRNA) which includes a region that is complementary or substantially complementary to a target sequence (e.g., a human phospholamban mRNA). The region that is “complementary” or “substantially complementary” need not be fully complementary to the target sequence and may have percent sequence identity to the target sequence of least 70%, 75%, 80%, 85%, 90%, 95%, or 100% due to, e.g., the presence of a mismatch region.

A “sense strand,” as used herein, refers to the strand of an interfering RNA (e.g., siRNA) that includes a region that is complementary or substantially complementary to a region of the antisense strand.

As used herein, the term “mismatch region” refers to a portion of an interfering RNA (e.g., siRNA) sequence that does not have 100% complementarity to its target sequence. An interfering RNA may have at least one, two, three, four, five, six, or more mismatch regions. The mismatch regions may be contiguous or may be separated by 1, 2, 3, 4, 5, 6, 7, 8, 9, 10, 11, 12, or more nucleotides. The mismatch region may comprise a single nucleotide or may comprise two, three, four, five, or more nucleotides.

“Percent sequence identity” or “percent identity” is determined by comparing two optimally aligned sequences over a comparison window, wherein the portion of the sequence (e.g., a polynucleotide of the invention) in the comparison window may comprise additions or deletions (i.e., gaps) as compared to the reference sequence that does not comprise additions or deletions, for optimal alignment of the two sequences. The percentage is calculated by determining the number of positions at which the identical nucleotide occurs in both sequences to yield the number of matched positions, dividing the number of matched positions by the total number of positions in the window of comparison and multiplying the result by 100 to yield the percentage of sequence identity.

The phrase “inhibiting expression of a target gene” refers to the ability of an interfering RNA (e.g., siRNA) described herein to silence, reduce, or inhibit the expression of a target gene (e.g., PLN). To examine the extent of gene silencing, a test sample (e.g., a biological sample from an organism of interest expressing the target gene or a sample of cells in culture expressing the target gene) is contacted with an interfering RNA (e.g., siRNA) that silences, reduces, or inhibits expression of the target gene. Expression of the target gene in the test sample is compared to expression of the target gene in a control sample (e.g., a biological sample from an organism of interest expressing the target gene or a sample of cells in culture expressing the target gene) that is not contacted with the interfering RNA (e.g., siRNA). Control samples (e.g., samples expressing the target gene) may be assigned a value of 100%. In particular embodiments, silencing, inhibition, or reduction of expression of a target gene is achieved when the value of the test sample relative to the control sample is about 95%, 90%, 85%, 80%, 75%, 70%, 65%, 60%, 55%, 50%, 45%, 40%, 35%, 30%, 25%, 20%, 10%, 5%, or 0%. Suitable assays include, without limitation, examination of protein or mRNA levels using techniques known to those of skill in the art, such as, e.g., dot blots, Northern blots, in situ hybridization, ELISA, immunoprecipitation, enzyme function, as well as phenotypic assays known to those of skill in the art.

As used herein, the term “knockdown” refers to a reduction in the expression level of the PLN gene. Knocking down PLN gene expression level may be achieved by reducing the amount of mRNA transcript corresponding to the gene, leading to a reduction in the expression level of PLN protein. A knockdown agent is an example of an inhibitor.

By “pharmaceutically acceptable,” it is meant that the excipient is compatible with the other ingredients of the formulation and is not deleterious to the recipient thereof.

A “patient” or “subject,” as used herein, is intended to include either a human or non-human animal, preferably a mammal, e.g., non-human primate. Most preferably, the subject or patient is a human.

When a subject is a “carrier of an allele” or an “allele carrier”, the subject has one or two genomic copies of the allele. For example, a carrier of the R14del allele (or R14del allele carrier, or R14del carrier) has one R14del mutant allele and one wild-type allele of PLN.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, can include treatment resulting in inhibiting the disease, i.e., arresting its development; and relieving the disease, i.e., causing regression of the disease. For example, in the case of dilated cardiomyopathy, a response to treatment can include complete response, partial response, stable disease, progressive disease, progression free survival, or overall survival.

An “effective amount” or “therapeutically effective amount” of an interfering RNA (e.g., siRNA) is an amount sufficient to produce the desired effect, e.g., an inhibition of expression of a target sequence in comparison to the normal expression level detected in the absence of an interfering RNA. In particular embodiments, inhibition of expression of a target gene or target sequence is achieved when the value obtained with an interfering RNA relative to the control is about 95%, 90%, 85%, 80%, 75%, 70%, 65%, 60%, 55%, 50%, 45%, 40%, 35%, 30%, 25%, 20%, 15%, 10%, 5%, or 0%. Suitable assays for measuring the expression of a target gene or target sequence include, but are not limited to, examination of protein or mRNA levels using techniques known to those of skill in the art, such as, e.g., dot blots, Northern blots, in situ hybridization, ELISA, immunoprecipitation, enzyme function, as well as phenotypic assays known to those of skill in the art.

The term “pharmaceutically acceptable carrier” refers to a substance that aids the administration of an active agent (e.g., an interfering RNA molecule) to a cell, an organism, or a subject. “Pharmaceutically acceptable carrier” refers to a carrier or excipient that can be included in the compositions of the disclosure and that causes no significant adverse toxicological effect on the subject. Non-limiting examples of pharmaceutically acceptable carriers include water, sodium chloride, normal saline solutions, lactated Ringer's, normal sucrose, normal glucose, binders, fillers, disintegrants, lubricants, coatings, sweeteners, flavors and colors, liposomes, dispersion media, microcapsules, cationic lipid carriers, isotonic and absorption delaying agents, and the like. The carrier may also be substances for providing the formulation with stability, sterility and isotonicity (e.g., antimicrobial preservatives, antioxidants, chelating agents and buffers), for preventing the action of microorganisms (e.g., antimicrobial and antifungal agents, such as parabens, chlorobutanol, sorbic acid and the like) or for providing the formulation with an edible flavor etc. One of skill in the art will recognize that other pharmaceutical carriers are useful in the present disclosure.

As used herein, the term “administering” or “administration” includes any route of introducing or delivering an agent that inhibits the expression or activity of PLN to a subject that is a carrier of the R14del allele. Administration can be carried out by any route suitable for the delivery of the agent. Thus, delivery routes can include, e.g., oral, intranasal, intravenous, intramuscular, intraperitoneal, intraarticular, intradermal, or subcutaneous.

As used herein, the terms “about” and “around” indicate a close range around a numerical value when used to modify that specific value. If “X” were the value, for example, “about X” or “around X” would indicate a value from 0.9X to 1.1X, e.g., a value from 0.95X to 1.05X, or a value from 0.98X to 1.02X, or a value from 0.99X to 1.01X. Any reference to “about X” or “around X” specifically indicates at least the values X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, and 1.1X, and values within this range.

### III. DESCRIPTION OF THE EMBODIMENTS

The present disclosure provides an interfering RNA molecule comprising a double-stranded region of about 15 to about 60 nucleotides in length, wherein the double-stranded region comprises a first nucleic acid strand and a second nucleic acid strand, wherein the interfering RNA molecule is capable of inhibiting expression of the R14del allele but not the wild-type allele of the phospholamban (PLN) gene.

One type of interfering RNA molecule is siRNA, also known as “small interfering RNA” or “short interfering RNA,” which comprises a double-stranded RNA where one strand is complementary or substantially complementary to the other strand. One strand is also complementary or substantially complementary to an mRNA sequence of interest that is a target for suppression.

An artificial miRNA, also known as an artificial microRNA or a synthetic miRNA, is another type of interfering RNA molecule that can be introduced into cells. An artificial miRNA can be a hairpin pre-miRNA precursor molecule that has been modified to replace the sequence encoding a mature miRNA with a sequence that encodes a different miRNA that can target an mRNA of interest and suppress its expression. See, e.g., Calloni and Bonatto, Scaffolds for Artificial miRNA Expression in Animal Cells, Hum Gene Ther Methods 26:162-74 (2015). The hairpin pre-miRNA precursor molecule can be >1000 nucleotides long. A mature miRNA is typically around 18 to 24 nucleotides long.

An artificial miRNA can be designed based on a fully complementary siRNA sequence, as described, for example, in Betancur et al. miRNA-like duplexes as RNAi triggers with improved specificity, Front Genet 3:127 (2012). For some artificial miRNA, a 5′ 6-8 nucleotide long seed sequence in the mature miRNA is fully complementary to a sequence in the target mRNA sequence (e.g., a sequence in the PLN R14del allele mRNA but not PLN WT allele mRNA). For some artificial miRNA, a 5′ 6-8 nucleotide long seed sequence in the mature miRNA is substantially complementary to a sequence in the target mRNA sequence (e.g., a sequence in the PLN R14del allele mRNA but not PLN WT allele mRNA), and may contain mismatches that enhance unwinding of the duplex and interaction between the mature miRNA and the RNA-induced silencing complex (RISC). Id.

In certain embodiments, the interfering RNA molecule comprises an siRNA molecule, an miRNA molecule, or a combination thereof.

In some embodiments, the second nucleic acid strand is substantially complementary to the first nucleic acid strand. In other embodiments, the second nucleic acid strand is fully complementary to the first nucleic acid strand. In some embodiments, mismatches within the double-stranded region promote duplex unwinding.

In some embodiments, the double-stranded region is 18 to 24 nucleotides in length. In some embodiments, the double-stranded region is about 20 to about 30 nucleotides in length. In some embodiments, the double-stranded region is about 25 nucleotides in length.

Example 2 illustrates a strategy that can be used to design and select interfering RNA molecules that selectively inhibit expression of R14del. These include siRNA 1, siRNA 2, and siRNA 3 used in the present disclosure. SEQ ID NO: 3 and SEQ ID NO: 4 are polynucleotide sequences found in the antisense and sense strand sequences, respectively, that are common to these three siRNAs.

In some embodiments, the first nucleic acid strand has at least 90% identity (e.g., at least 90%, 91%, 92%, 93%, 94%, 95%, 96%, 97%, 98%, 99%, or 100% identity) to SEQ ID NO: 3. In some embodiments, the first nucleic acid strand comprises SEQ ID NO: 3. In some embodiments, the second nucleic acid strand has at least 90% identity (e.g., at least 90%, 91%, 92%, 93%, 94%, 95%, 96%, 97%, 98%, 99%, or 100% identity) to SEQ ID NO: 4. In some embodiments, the second nucleic acid strand comprises SEQ ID NO: 4.

SEQ ID NO: 5 and SEQ ID NO: 6 are polynucleotide sequences found in the antisense and sense strand sequences, respectively, of siRNA 1. In some embodiments, the first nucleic acid strand has at least 90% identity (e.g., at least 90%, 91%, 92%, 93%, 94%, 95%, 96%, 97%, 98%, 99%, or 100% identity) to SEQ ID NO: 5. In some embodiments, the first nucleic acid strand comprises SEQ ID NO: 5. In some embodiments, the first nucleic acid strand has at least 90% identity (e.g., at least 90%, 91%, 92%, 93%, 94%, 95%, 96%, 97%, 98%, 99%, or 100% identity) to SEQ ID NO: 6.

SEQ ID NO: 7 and SEQ ID NO: 8 are polynucleotide sequences found in the antisense and sense strand sequences, respectively, of siRNA 2. In some embodiments, the first nucleic acid strand has at least 90% identity (e.g., at least 90%, 91%, 92%, 93%, 94%, 95%, 96%, 97%, 98%, 99%, or 100% identity) to SEQ ID NO: 7. In some embodiments, the first nucleic acid strand comprises SEQ ID NO: 7. In some embodiments, the first nucleic acid strand has at least 90% identity (e.g., at least 90%, 91%, 92%, 93%, 94%, 95%, 96%, 97%, 98%, 99%, or 100% identity) to SEQ ID NO: 8.

SEQ ID NO: 9 and SEQ ID NO: 10 are polynucleotide sequences found in the antisense and sense strand sequences, respectively, of siRNA 3. In some embodiments, the first nucleic acid strand has at least 90% identity (e.g., at least 90%, 91%, 92%, 93%, 94%, 95%, 96%, 97%, 98%, 99%, or 100% identity) to SEQ ID NO: 9. In some embodiments, the first nucleic acid strand comprises SEQ ID NO: 9. In some embodiments, the first nucleic acid strand has at least 90% identity (e.g., at least 90%, 91%, 92%, 93%, 94%, 95%, 96%, 97%, 98%, 99%, or 100% identity) to SEQ ID NO: 10. In some embodiments, the first nucleic acid strand comprises SEQ ID NO: 10.

As shown in Examples 3, 4, and 5, RNA interfering molecule-mediated knockdown of phospholamban in cardiac muscle cells derived from heterozygous patient carriers can be highly allele-specific, resulting in a significant increase in the WT/R14del allele ratio.

In some embodiments, the interfering RNA molecule described herein includes optional 3′ overhangs in a nucleic acid strand and/or incorporation of modified nucleotides (see, e.g., Selvam et al., Recent Trends Chem. Biol. Drug Des., 90: 665-78 (2017)). In particular embodiments, the interfering RNA molecule comprises a 3′ overhang in the first nucleic acid strand and/or the second nucleic acid strand. In particular embodiments, one or more of the nucleotides in the double-stranded region comprise modified nucleotides.

In some embodiments, the first nucleic acid strand or the second nucleic acid strand comprises a 5′ 6 to 8 nucleotide long sequence fully complementary to a sequence in the R14del allele mRNA but not WT allele mRNA of PLN. In some embodiments, the first nucleic acid strand or the second nucleic acid strand comprises a 5′ 6 to 8 nucleotide long sequence substantially complementary to a sequence in the R14del allele mRNA but not WT allele mRNA of PLN.

Interfering RNA molecules are activated to suppress gene expression in the cytoplasm. In order to reach these areas or target cells, interfering RNA molecules must face several challenges, including stability against serum nucleases, avoiding immune recognition, reaching the target cells without being cleared, and entering target cells while effectively escaping endosomes and lysosomes. In some embodiments, an interfering RNA molecule without a carrier system may be used, and in vivo delivery relies upon modifications to enable the interfering RNA molecule to be resistant to enzyme degradation and/or targeted local injection.

In some embodiments, the interfering RNA molecule further comprises a carrier system. Also provided is a pharmaceutical composition comprising a carrier system to deliver the interfering RNA molecule to a target cell where it can inhibit gene expression. The carrier system may comprise lipid or lipidoid nanoparticles that protect the nucleic acid payload (e.g., siRNA) from, for example, nuclease attack and renal clearance. Systemic delivery for in vivo therapy, e.g., delivery of a nucleic acid to a distal target cell via body systems such as the circulation, has been achieved using nucleic acid-lipid particles such as those described in PCT Publication Nos. WO 05/007196, WO 05/121348, WO 05/120152, and WO 04/002453, the disclosures of which are herein incorporated by reference in their entirety for all purposes. In some embodiments, the interfering RNA molecule is delivered using fully encapsulated lipid particles that protect the nucleic acid (e.g., siRNA) from nuclease degradation in serum, are non-immunogenic, are small in size, and are suitable for repeat dosing.

In some embodiments, the carrier system may comprise DPC™ and TRIM™ delivery platforms that use a polymer backbone attached to the interfering RNA, a shielding agent, and targeting ligand, and/or a cholesterol modification on the interfering RNA. The carrier system may comprise Local Drug Eluter (LODERTM™) and/or siG12D-LODERTM™ systems that use a biodegradable polymer matrix. The carrier system may comprise other systems as described in Hu et al., Signal Transduction and Targeted Therapy, 5:101 (2020), such as the polymers PEI, PTMs, GDDC4, and PAsp (DET).

### In Vivo Administration

For in vivo administration, administration can be in any manner known in the art, e.g., by injection, oral administration, inhalation (e.g., intransal or intratracheal), transdermal application, or rectal administration. Administration can be accomplished via single or divided doses. The pharmaceutical compositions can be administered parenterally, i.e., intraarticularly, intravenously, intraperitoneally, subcutaneously, or intramuscularly. In some embodiments, the pharmaceutical compositions are administered intravenously or intraperitoneally by a bolus injection (see, e.g., U.S. Pat. No. 5,286,634). Intracellular nucleic acid delivery has also been discussed in Straubringer et al., Methods Enzymol., 101:512 (1983); Mannino et al., Biotechniques, 6:682 (1988); Nicolau et al., Crit. Rev. Ther. Drug Carrier Syst., 6:239 (1989); and Behr, Acc. Chem. Res., 26:274 (1993).

The pharmaceutical compositions described herein, either alone or in combination with other suitable components, can be made into aerosol formulations (i.e., they can be “nebulized”) to be administered via inhalation (e.g., intranasally or intratrachcally) (see, Brigham et al., Am. J. Sci., 298:278 (1989)). Aerosol formulations can be placed into pressurized acceptable propellants, such as dichlorodifluoromethane, propane, nitrogen, and the like.

In certain embodiments, the pharmaceutical compositions may be delivered by intranasal sprays, inhalation, and/or other aerosol delivery vehicles. Methods for delivering nucleic acid compositions directly to the lungs via nasal aerosol sprays have been described, e.g., in U.S. Pat. Nos. 5,756,353 and 5,804,212. Likewise, the delivery of drugs using intranasal microparticle resins and lysophosphatidyl-glycerol compounds (U.S. Pat. No. 5,725,871) are also well-known in the pharmaceutical arts. Similarly, transmucosal drug delivery in the form of a polytetrafluoroetheylene support matrix is described in U.S. Pat. No. 5,780,045. The disclosures of the above-described patents are herein incorporated by reference in their entirety for all purposes.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions are preferably administered, for example, by intravenous infusion, orally, topically, intraperitoneally, intravesically, or intrathecally.

Generally, when administered intravenously, the pharmaceutical compositions are formulated with a pharmaceutically acceptable carrier (or carrier system). Many pharmaceutically acceptable carriers may be employed in the compositions and methods of the present invention. A pharmaceutically acceptable carrier may contain one or more excipients. An excipient is a substance that aids the administration of an active agent to a subject. Pharmaceutical excipients useful in the compositions include, but are not limited to, binders, fillers, disintegrants, lubricants, glidants, coatings, sweeteners, flavors and colors. Suitable formulations are found, for example, in REMINGTON's PHARMACEUTICAL SCIENCES, Mack Publishing Company, Philadelphia, PA, 17th ed. (1985). A variety of aqueous carriers may be used, for example, water, buffered water, 0.4% saline, 0.3% glycine, and the like, and may include glycoproteins for enhanced stability, such as albumin, lipoprotein, globulin, etc. Generally, normal buffered saline (135-150 mM NaCl) will be employed as the pharmaceutically acceptable carrier, but other suitable carriers will suffice. These compositions can be sterilized by conventional liposomal sterilization techniques, such as filtration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc. These compositions can be sterilized using the techniques referred to above or, alternatively, they can be produced under sterile conditions. The resulting aqueous solutions may be packaged for use or filtered under aseptic conditions and lyophilized, the lyophilized preparation being combined with a sterile aqueous solution prior to administration.

In certain applications, the pharmaceutical compositions disclosed herein may be delivered via oral administration to the individual. The pharmaceutical compositions may be incorporated with excipients and used in the form of ingestible tablets, buccal tablets, troches, capsules, pills, lozenges, elixirs, mouthwash, suspensions, oral sprays, syrups, wafers, and the like (see, e.g., U.S. Pat. Nos. 5,641,515, 5,580,579, and 5,792,451, the disclosures of which are herein incorporated by reference in their entirety for all purposes). These oral dosage forms may also contain the following: binders, gelatin; excipients, lubricants, and/or flavoring agents. When the unit dosage form is a capsule, it may contain, in addition to the materials described above, a liquid carrier. Various other materials may be present as coatings or to otherwise modify the physical form of the dosage unit. Of course, any material used in preparing any unit dosage form should be pharmaceutically pure and substantially non-toxic in the amounts employed.

Naturally, the amount of pharmaceutical composition in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

Formulations suitable for oral administration can consist of: (a) liquid solutions, such as an effective amount of a packaged therapeutic nucleic acid (e.g., interfering RNA) suspended in diluents such as water, saline, or PEG 400; (b) capsules, sachets, or tablets, each containing a predetermined amount of a therapeutic nucleic acid (e.g., interfering RNA), as liquids, solids, granules, or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise a therapeutic nucleic acid (e.g., interfering RNA) in a flavor, e.g., sucrose, as well as pastilles comprising the therapeutic nucleic acid in an inert base, such as gelatin and glycerin or sucrose and acacia emulsions, gels, and the like containing, in addition to the therapeutic nucleic acid, carriers known in the art.

The amount of pharmaceutical composition administered will depend upon the ratio of therapeutic nucleic acid (e.g., interfering RNA) to carrier, the particular therapeutic nucleic acid used, the disease or disorder being treated, the age, weight, and condition of the patient, and the judgment of the clinician.

### In Vitro Administration

For in vitro applications, the delivery of therapeutic nucleic acids (e.g., interfering RNA) can be to any cell grown in culture, whether of plant or animal origin, vertebrate or invertebrate, and of any tissue or type. In particular embodiments, the cells are animal cells, e.g., mammalian cells such as human cells.

Contact between the cells and the therapeutic nucleic acids (e.g., interfering RNA), when carried out in vitro, takes place in a biologically compatible medium. To the extent that tissue culture of cells may be required, it is well-known in the art. For example, Freshney, Culture of Animal Cells, a Manual of Basic Technique, 3rd Ed., Wiley-Liss, New York (1994), Kuchler et al., Biochemical Methods in Cell Culture and Virology, Dowden, Hutchinson and Ross, Inc. (1977), and the references cited therein provide a general guide to the culture of cells. Cultured cell systems often will be in the form of monolayers of cells, although cell suspensions are also used.

The present disclosure also provides a pharmaceutical composition comprising any siRNA molecule described herein and a pharmaceutically acceptable carrier.

The present disclosure also provides a method for introducing an interfering RNA molecule that is capable of inhibiting the expression of the R14del allele of the PLN gene in a cell, wherein the method comprises contacting the cell with any interfering RNA molecule described herein or any pharmaceutical composition described herein. In some embodiments, the cell is a cardiac muscle cell. In some embodiments, the cell is in a subject. In some embodiments, the subject is a carrier of the R14del allele of the PLN gene. In some embodiments, the subject has been diagnosed with cardiomyopathy. In some embodiments, the subject is a human.

In some embodiments, the interfering RNA molecule does not affect the contractility and/or action potential of a cardiac muscle cell. As shown in Example 6, the siRNAs tested had no apparent negative effects on either the contractility or action potential of iPSC-CMs. In some embodiments, the interfering RNA molecule has a positive effect on the contractility and/or action potential of a cardiac muscle cell. Such a positive effect could include an improvement in the regularity of the waveform, the duration of the action potential, and/or the amplitude of the action potential.

The present disclosure also provides a method for in vivo delivery of an interfering RNA molecule that is capable of inhibiting the expression of the R14del allele of the PLN gene in a cell of a subject, the method comprising administering to the subject any siRNA molecule described herein or any pharmaceutical composition described herein. In some embodiments, the cell is a cardiac muscle cell. In some embodiments, the subject is a carrier of the R14del allele of the PLN gene. In some embodiments, the subject has been diagnosed with cardiomyopathy. In some embodiments, the subject is a human.

The present disclosure also provides a method for treating cardiomyopathy in a subject carrying the R14del allele of the PLN gene, the method comprising administering to the subject a therapeutically effective amount of any interfering RNA molecule described herein or any pharmaceutical composition described herein.

The present disclosure also provides a method for lowering the likelihood of at least one of malignant arrhythmias, sudden cardiac death, and/or a heart transplant in a subject carrying the R14del allele of the PLN gene, the method comprising administering to the subject a therapeutically effective amount of any interfering RNA molecule described herein or any pharmaceutical composition described herein.

In some embodiments, the administration is selected from the group consisting of oral, intranasal, intravenous, intraperitoneal, intramuscular, intraarticular, intralesional, subcutaneous, and intradermal. In some embodiments, the subject has been diagnosed with cardiomyopathy. In some embodiments, the subject is a human.

### IV. EXAMPLES

The subject matter of the present disclosure will be described in greater detail by way of specific examples. The following examples are provided to illustrate, but not to limit, the claimed subject matter.

### Example 1

### Materials and Methods

Specific oligonucleotide sequences used. SEQ ID NO: 1 and SEQ ID NO: 2 show reference sequences used to design small-interfering RNAs (siRNAs) that target the R14del mutant allele but not the wild-type (WT) allele of PLN. siRNAs were designed as 27-mer duplexes based on Kim, D H., et al. (Nat Biotechnol. 2005; 23, 222-226). We used the PLN mutant sequence as a reference that includes a 3 bp deletion in exon 2 of the PLN gene [NM_002667.3 (PLN): c.40_42delAGA (p.R14del)].

Measuring allele-specific mRNA knockdown. Patient-specific induced pluripotent stem cells (iPSCs) carrying a heterozygous PLN c.40_42delAGA (p.R14del) were differentiated into cardiomyocytes. Thirty days post differentiation, iPSC-cardiomyocytes (1.0×10⁶) were seeded in 6-well plates in antibiotic-free medium and transfected with 30 pmol of siRNA (siRNA 1, siRNA 2, siRNA 3 or non-targeting control siRNA) and 9 μl RNAimax reagent (Thermofisher Scientific). In parallel, iPSC-cardiomyocytes derived from a healthy donor (WT PLN) were transfected with siRNA as controls.

At 96 h after the transfections, cells were collected for RNA extraction. Total RNA was extracted with the RNeasy kit (Qiagen), and the cDNA was synthesized with a random primer with the MultiScribe reverse transcriptase kit (ThermoFisher Scientific). Total PLN expression was assessed by real time quantitative PCR detection using a PLN specific TaqMan probes. The reactions were run in 2 biological and 4 technical replicates per line in an QuantStudio 7 Flex system (Applied Biosystems). The results were analyzed by the 2-ANACT method normalized to 18 s endogenous control.

Contractility measurements. The iPSC-CMs were seeded in a 384-well plate at a cell density of 2×10⁴ cells per well and transfected with siRNAs (10 nM duplex concentration) using Lipofectamine RNAimax reagent (Thermo Fisher Scientific). After 2 days the iPSC-CMs were stained with tetramethylrhodamine, methyl ester (TMRM) (Marker Gene Technologies) diluted in RPMI 1640 medium supplemented with B27 at a final concentration of 400 nM to stain the mitochondria. Fluorescent high-speed movies were recorded from each well using the IC200 Kinetic Imaging Cytometer (Vala Sciences) at an acquisition frequency of 100 Hz at 20× magnification for a duration of 10 seconds. Contractility was assessed by analyzing the movies using a custom particle image velocity software.

Action Potential measurements. The iPSC-CMs were seeded in a 384-well plate at a cell density of 2×10⁴ cells per well and transfected with siRNAs (10 nM duplex concentration) using Lipofectamine RNAimax reagent (Thermo Fisher Scientific). After four days the iPSC-CMs were incubated with the VF2.1.Cl voltage-sensitive dye (Life Technologies) diluted FluoroBrite DMEM media (Gibco) for 50 min at 37° C. After incubation, cells were washed four times in FluoroBrite and time series images were acquired automatically using the IC200 KIC instrument (Vala Sciences) at an acquisition frequency of 100 Hz for a duration of 10 seconds. The voltage image analysis and physiological parameter calculation were conducted using commercially available Cyteseer software (Vala Sciences).

Transcription Profiling. Sequencing libraries were generated using the NEBNext Ultra Directional RNA Library Prep Kit for Illumina. Clustering of the index-coded samples was performed on a eBot Cluster Generation System using TruSeq PE Cluster Kit v3-cBot-HS (Illumina). Sequencing was carried out on an Illumina HiSeq platform. For each sample in the whole transcriptome sequencing library, 30 to 40 million 150-base pair paired-end reads were acquired. Raw sequence data were checked for quality with FastQC (version 0.11.5), and results were aggregated with MultiQC. Sequence data were aligned to the human genome (hg38) using STAR (version 2.5.1b) with ENCODE options for long RNA-seq pipeline. Alignment results were assessed using Samtools and aggregated with MultiQC (version 0.9). Uniquely mapped reads were quantified using featureCounts (version 1.28.0).

### Example 2

### Allele-Specific siRNA-Based Approach

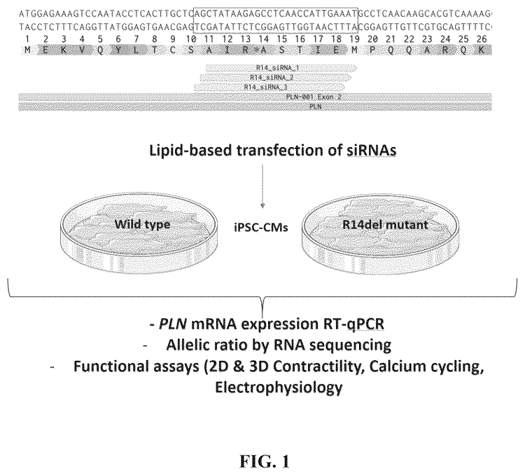

siRNAs that target R14del phospholamban allele transcripts but not WT phospholamban allele transcripts in R14del iPSC-CMs were designed as shown in FIG. 1. iPSC-CMs containing only WT phospholamban were used in control experiments. After transfection, PLN mRNA expression was monitored, as well as the ratios of WT and R14del alleles. Functional assays were then performed to assess contractility and electrophysiology.

### Example 3

### siRNA-Mediated Knockdown of PLN R14del mRNA in iPSC-Derived Cardiomyocytes (iPSC-CMs)

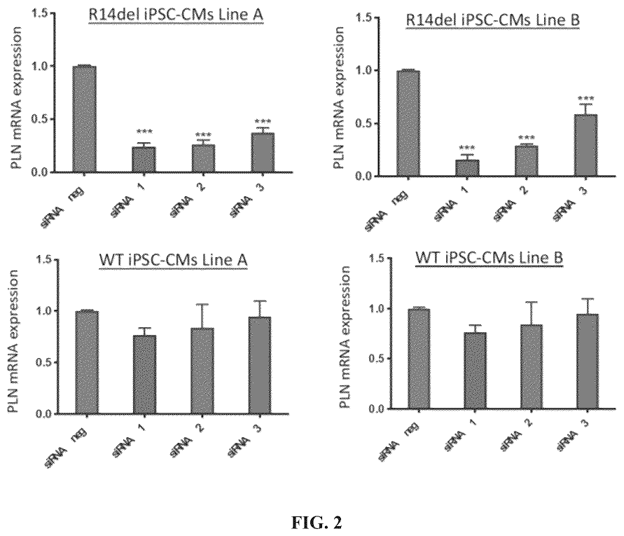

A significant decrease in the expression of PLN mRNA was observed in PLN R14del cardiomyocytes treated with siRNAs 1, 2 and 3 compared to negative control siRNA-treated cells (FIG. 2, top panels). In contrast, treatment with siRNAs 1, 2, and 3 had little effect on the PLN mRNA expression in WT control iPSC-derived cardiomyocytes (FIG. 2, bottom panels). These data demonstrate that siRNAs 1, 2, and 3 that target the R14del allele inhibited the expression of PLN mutant alleles without suppressing the expression of the corresponding WT PLN allele.

### Example 4

### siRNAs Targeting the R14del Allele Exhibit Allele-Specific Silencing Properties in Patient-Specific iPSC-CMs

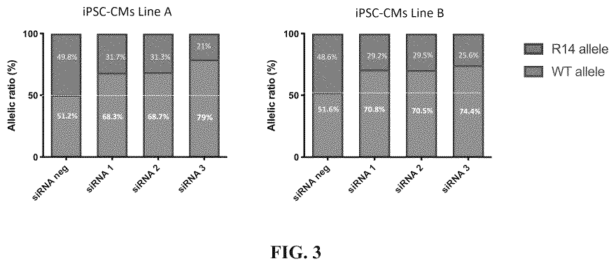

The abundance of mutant and WT PLN alleles in R14del iPSC-CMs was measured following treatment with siRNA 1, 2, 3, or negative control in PLN R14del iPSC cardiomyocytes. The PLN region flanging the mutation was PCR amplified and the allele abundance was assessed by next generation amplicon sequencing. As shown in FIG. 3, in control siRNA-treated iPSC-CMs, a mutant/WT ratio equal to ˜1 was observed, in agreement with similar expression of both WT and mutant alleles in PLN R 14del IPSC-CMs. In contrast, siRNAs 1, 2, and 3 that targeted the R14del allele exhibited allele-specific silencing properties as demonstrated by a significant reduction in mutant/WT ratios (˜1:3-1:4 ratio) compared to control siRNA-transfected cells (FIG. 3).

### Example 5

### RNAseq Analysis of PLN R14del iPSC-CMs Transfected With siRNA 3 Show a Significant Decrease in the Expression of the Mutant Allele and a High WT/R14del Allele Ratio

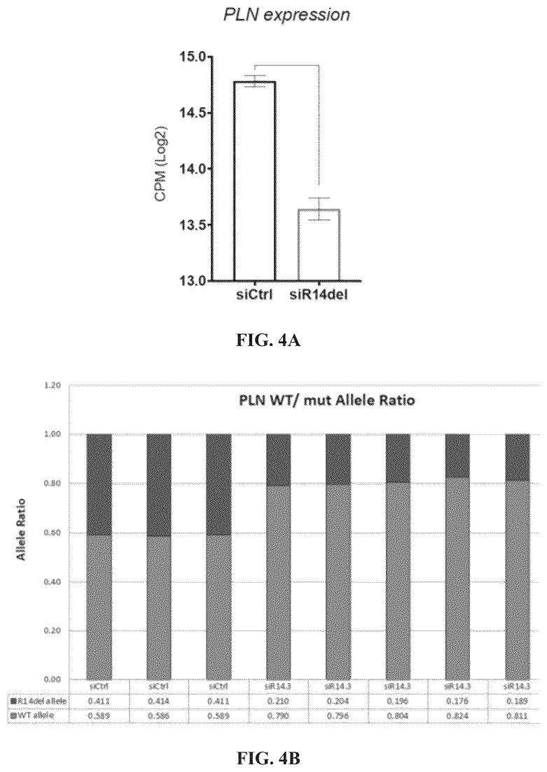

Whole transcriptome RNAseq was used to validate the allele-specific silencing properties of siRNA 3 and to assess the ratio of WT to mutant PLN. FIG. 4A shows that there is an absolute difference in the counts per million reads (CPM) as measure of PLN expression when iPSC-CMs are treated with siRNA 3 versus an siRNA control.

As shown in FIG. 4B, the ratio of WT to mutant alleles in iPSC-CMs carrying a heterozygous PLN (p.R14del) was around 60% when cells were transfected with a control siRNA (right hand, columns 1-3), whereas the ratio in cells transfected with siR14.3 was around 80% (left hand, columns 4-8).

### Example 6

### No Adverse Functional Effects of siRNA Mediated R14del Silencing In Vitro

High throughput optical contractility assay and high throughput optical voltage assay were performed to investigate if there are any adverse functional effects of siRNA-mediated R14del silencing in iPSC-CMs.

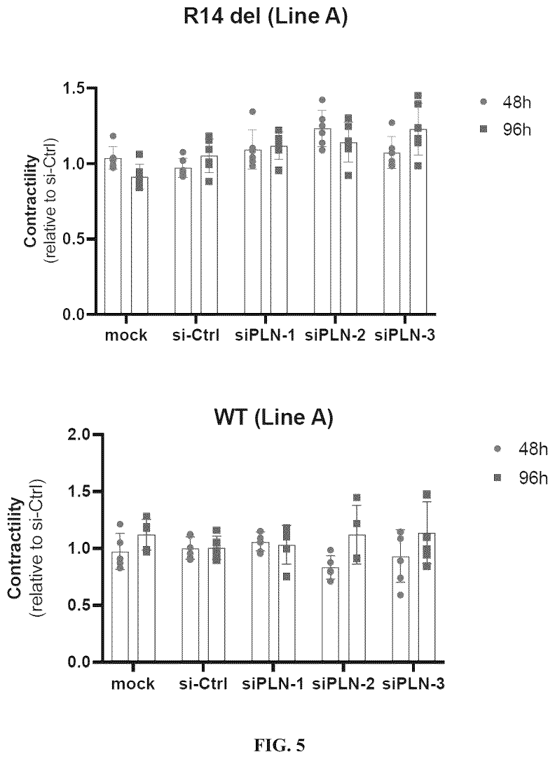

For the high throughput optical contractility assay, both WT and PLN R14del hiPSC-CMs were separately plated on Matrigel at 25,000 cells per well of a 384-well plate and transfected with 5 nM of siRNAs 1, 2, 3, or non-targeting control siRNA (si-Ctrl) or without a siRNA (mock) using Lipofectamine RNAimax transfection reagent. After 48 or 96 hours, the cells were washed with FluoroBrite DMEM and labelled with tetramethylrhodamine methyl ester dye. Time-series images were then automatically acquired using an IC200 KIC instrument (Vala Sciences) at an acquisition frequency of 33 Hz for a duration of 10 seconds. The image analysis and contractility parameters were calculated using custom particle image velocity software. As shown in FIG. 5, the force of contraction was comparable between neg siRNA and siRNA-treated WT iPSC-CMs, and between neg siRNA and siRNA-treated PLN R14del iPSC-CMs. These findings indicate that knockdown of the PLN R14del mutant allele does not have a detrimental effect on the physiology of the cells.

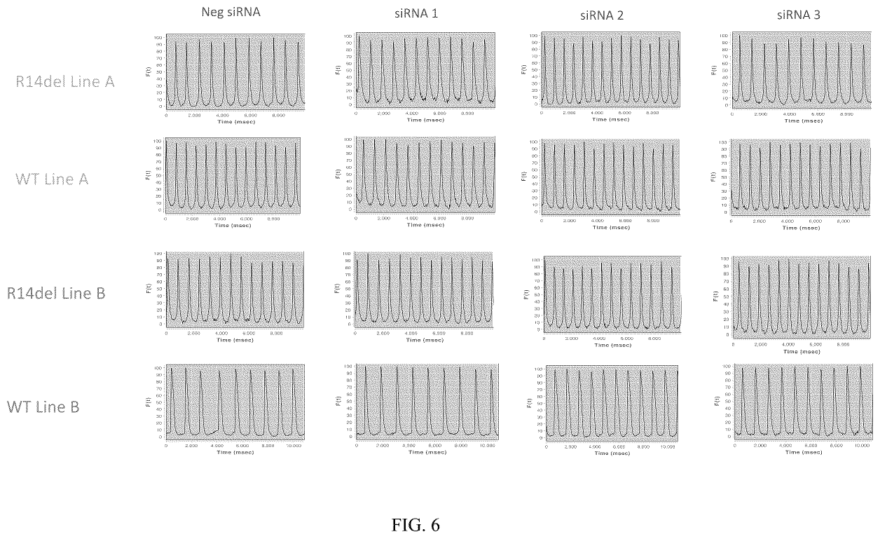

For the high throughput optical voltage assay, both WT or PLN R14del hiPSC-CMs were plated separately on Matrigel at 25,000 cells per well of a 384-well plate and transfected with 5nM of siRNAs 1, 2, 3, or non-targeting control siRNA (Neg siRNA) using Lipofectamine RNAimax transfection reagent. After 96 hours, the cells were washed with FluoroBrite DMEM and loaded with the VF2.1.Cl dye with Hoechst 33258 (Life Technologies) by incubation at 37° C. for 50 minutes. After incubation, the cells were washed four times in FluoroBrite DMEM media. Time series images were then acquired automatically using the IC200 KIC instrument (Vala Sciences) at an acquisition frequency of 33 Hz for a duration of 10 seconds. The voltage image analysis and electrophysiological parameter calculation were conducted using the Cyteseer software (Vala Sciences). As shown in FIG. 6, the action potential waveforms were normal and comparable between the neg-siRNA and siRNAs-treated WT iPSC-CMs, and between the neg-siRNA and siRNA-treated PLN R14del iPSC-CMs. These findings indicate that the knockdown of the PLN R14del mutant allele does not have a detrimental effect on the electrophysiological properties of the cells.

These findings demonstrate that there was no significant effect of siRNA 1, 2, or 3 on contractility and action potential of the iPSC-CMs.

### Example 7

### Treatment With siRNA 3 Restores Contractility in Engineered Heart Tissues (EHTs) Generated From PLN R14del iPSC-CMs

Skin fibroblasts from a patient carrying the PLN R14del allele were reprogrammed to human iPSCs using the CytoTuncTM-iPS 2.0 Sendai Reprogramming Kit. The hiPSCs were differentiated to cardiomyocytes using a small molecule Wnt-activation/inhibition protocol.

Three-dimensional engineered heart tissues (3D-EHTs) were generated in agarose casting molds using solid silicone racks (EHT Technologies). Briefly, about 1×10⁶ iPSC-CMs were suspended in a fibrin hydrogel (100 μL total) composed of 10 μL Matrigel (Corning), 5 mg/mL bovine fibrinogen supplemented with 0.1 mg/mL aprotinin (Sigma Aldrich) and 3 U/mL thrombin (Sigma Aldrich). siRNA 3 or an siRNA control (5 nM) was mixed with Lipofectamine RNAimax transfection reagent and added in the hydrogel. Once polymerized, the silicone racks with the newly formed fibrin gels were cultured for 4 weeks in culture medium consisting of DMEM: RPMI media (1:1) supplemented with 0.25% dialyzed fetal bovine serum (JR Scientific), 0.5×B27 supplement (Gibco), 5% knock-out serum replacement (Gibco).

Contractility measurements were performed via video recording. Videos of the deflecting posts were recorded at 75 frames/s at 4-weeks after the EHTs were made. The video recordings were processed by a custom ImageJ tool and the absolute force values were derived from calibrated measurements of post displacement using a custom Python script by considering an elastic modulus of 1.7 MPa, a post radius of 0.5 mm and a distance between posts (length) of 10 mm.

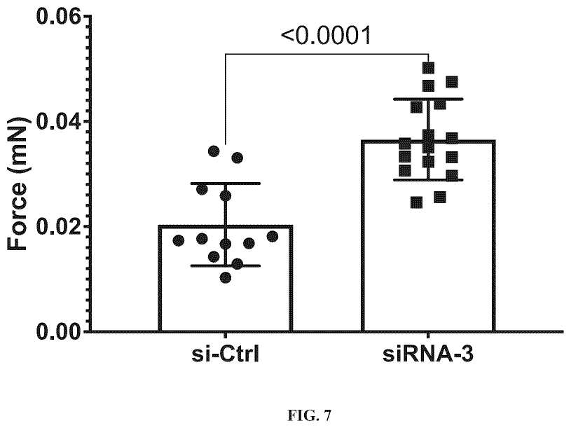

A significant increase was observed in the contractile force of the EHTs treated with siRNA 3 compared to the siRNA control (FIG. 7), demonstrating that the allelic-specific knockdown of the PLN R14del mutant allele can restore the contractile deficit associated with the R14del-caused dilated cardiomyopathy (DCM).

### V. EXEMPLARY EMBODIMENTS

Exemplary embodiments provided in accordance with the presently disclosed subject matter include, but are not limited to, the claims and the following embodiments:

Embodiment 1. An interfering RNA molecule comprising a double-stranded region of about 15 to about 60 nucleotides in length, wherein the double-stranded region comprises a first nucleic acid strand and a second nucleic acid strand, wherein the interfering RNA molecule is capable of inhibiting expression of the R14del allele but not the wild-type allele of the phospholamban (PLN) gene.

Embodiment 2. The interfering RNA molecule of embodiment 1, wherein the interfering RNA molecule comprises an siRNA molecule, an artificial miRNA molecule, or a combination thereof.

Embodiment 3. The interfering RNA molecule of embodiment 1 or 2, wherein the second nucleic acid strand is substantially complementary to the first nucleic acid strand.

Embodiment 4. The interfering RNA molecule of any one of embodiments 1 to 3, wherein the second nucleic acid strand is fully complementary to the first nucleic acid strand.

Embodiment 5. The interfering RNA molecule of any one of embodiments 1 to 3, wherein mismatches in the double-stranded region promote duplex unwinding.

Embodiment 6. The interfering RNA molecule of any one of embodiments 1 to 5, wherein the double-stranded region is about 20 to about 30 nucleotides in length.

Embodiment 7. The interfering RNA molecule of any one of embodiments 1 to 6, wherein the double-stranded region is about 25 nucleotides in length.

Embodiment 8. The interfering RNA molecule of any one of embodiments 1 to 3, wherein the first nucleic acid strand has at least 90% identity to SEQ ID NO: 3.

Embodiment 9. The interfering RNA molecule of embodiment 8, wherein the first nucleic acid strand comprises SEQ ID NO: 3.

Embodiment 10. The interfering RNA molecule of embodiment 8 or 9, wherein the second nucleic acid strand has at least 90% identity to SEQ ID NO: 4.

Embodiment 11. The interfering RNA molecule of embodiment 10, wherein the second nucleic acid strand comprises SEQ ID NO: 4.

Embodiment 12. The interfering RNA molecule of any one of embodiments 1 to 3, wherein the first nucleic acid strand has at least 90% identity to SEQ ID NO: 5.

Embodiment 13. The interfering RNA molecule of embodiment 12, wherein the first nucleic acid strand comprises SEQ ID NO: 5.

Embodiment 14. The interfering RNA molecule of embodiment 12 or 13, wherein the second nucleic acid strand has at least 90% identity to SEQ ID NO: 6.

Embodiment 15. The interfering RNA molecule of embodiment 14, wherein the second nucleic acid strand comprises SEQ ID NO: 6.

Embodiment 16. The interfering RNA molecule of any one of embodiments 1 to 3, wherein the first nucleic acid strand has at least 90% identity to SEQ ID NO: 7.

Embodiment 17. The interfering RNA molecule of embodiment 16, wherein the first nucleic acid strand comprises SEQ ID NO: 7.

Embodiment 18. The interfering RNA molecule of embodiment 16 or 17, wherein the second nucleic acid strand has at least 90% identity to SEQ ID NO: 8.

Embodiment 19. The interfering RNA molecule of embodiment 18, wherein the second nucleic acid strand comprises SEQ ID NO: 8.

Embodiment 20. The interfering RNA molecule of any one of embodiments 1 to 3, wherein the first nucleic acid strand has at least 90% identity to SEQ ID NO: 9.

Embodiment 21. The interfering RNA molecule of embodiment 20, wherein the first nucleic acid strand comprises SEQ ID NO: 9.

Embodiment 22. The interfering RNA molecule of embodiment 20 or 21, wherein the second nucleic acid strand has at least 90% identity to SEQ ID NO: 10.

Embodiment 23. The interfering RNA molecule of embodiment 22, wherein the second nucleic acid strand comprises SEQ ID NO: 10.

Embodiment 24. The interfering RNA molecule of any one of embodiments 1 to 23, wherein the interfering RNA molecule comprises a 3′ overhang in the first nucleic acid strand and/or the second nucleic acid strand.

Embodiment 25. The interfering RNA molecule of any one of embodiments 1 to 24, wherein one or more of the nucleotides in the double-stranded region comprise modified nucleotides.

Embodiment 26. The interfering RNA molecule of any one of embodiments 1 to 25, further comprising a carrier system.

Embodiment 27. A pharmaceutical composition comprising an interfering RNA molecule of any one of embodiments 1 to 26 and a pharmaceutically acceptable carrier.

Embodiment 28. A method for introducing an interfering RNA molecule that is capable of inhibiting expression of the R14del allele of the PLN gene in a cell, the method comprising:
contacting the cell with the interfering RNA molecule of any one of embodiments 1 to 26 or with the pharmaceutical composition of embodiment 27.

Embodiment 29. The method of embodiment 28, wherein the cell is a cardiac muscle cell.

Embodiment 30. The method of embodiment 29, wherein the cell is a cardiomyocyte.

Embodiment 31. The method of any one of embodiments 28 to 30, wherein the cell is in a subject.

Embodiment 32. The method of embodiment 31, wherein the subject is a carrier of the R14del allele of the PLN gene.

Embodiment 33. The method of embodiment 31 or 32, wherein the subject has been diagnosed with cardiomyopathy.

Embodiment 34. The method of any one of embodiments 31 to 33, wherein the subject is a human.

Embodiment 35. The method of any one of embodiments 28 to 34, wherein the interfering RNA molecule does not affect the contractility and/or the action potential of the cardiac muscle cell.

Embodiment 36. The method of any one of embodiments 28 to 34, wherein the interfering RNA molecule has a positive effect on the contractility and/or the action potential of the cardiac muscle cell.

Embodiment 37. A method for in vivo delivery of an interfering RNA molecule that is capable of inhibiting expression of the R14del allele of the PLN gene in a cell of a subject, the method comprising:
administering to the subject the interfering RNA molecule of any one of embodiments 1 to 26 or the pharmaceutical composition of embodiment 27.

Embodiment 38. The method of embodiment 37, wherein the cell is a cardiac muscle cell.

Embodiment 39. The method of embodiment 37 or 38, wherein the subject is a carrier of the R14del allele of the PLN gene.

Embodiment 40. The method of any one of embodiments 37 to 39, wherein the subject has been diagnosed with cardiomyopathy.

Embodiment 41. The method of any one of embodiments 37 to 40, wherein the subject is a human.

Embodiment 42. A method for treating cardiomyopathy in a subject carrying the R14del allele of the PLN gene, the method comprising:
administering to the subject a therapeutically effective amount of the interfering RNA molecule of any one of embodiments 1 to 26 or the pharmaceutical composition of embodiment 27.

Embodiment 43. A method for lowering the likelihood of at least one of malignant arrhythmias, sudden cardiac death, and/or a heart transplant in a subject carrying the R14del allele of the PLN gene, the method comprising:
administering to the subject a therapeutically effective amount of the interfering RNA molecule of any one of embodiments 1 to 26 or the pharmaceutical composition of embodiment 27.

Embodiment 44. The method of embodiment 42 or 43, wherein the subject has been diagnosed with cardiomyopathy.

Embodiment 45. The method of any one of embodiments 42 to 44, wherein the subject is a human.

Embodiment 46. The method of any one of embodiments 37 to 45, wherein the mode of administration is selected from the group consisting of oral, intranasal, intravenous, intraperitoneal, intramuscular, intraarticular, intralesional, subcutaneous, and intradermal.

Although the foregoing disclosure has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

What is claimed is:

1. An interfering RNA molecule comprising a double-stranded region of about 15 to about 60 nucleotides in length, wherein the double-stranded region comprises a first nucleic acid strand and a second nucleic acid strand, wherein the interfering RNA molecule is capable of inhibiting expression of the R14del allele but not the wild-type allele of the phospholamban (PLN) gene.

2. The interfering RNA molecule of claim 1, wherein the interfering RNA molecule comprises an siRNA molecule, an artificial miRNA molecule, or a combination thereof.

3. The interfering RNA molecule of claim 1 or 2, wherein the second nucleic acid strand is substantially complementary to the first nucleic acid strand.

4. The interfering RNA molecule of any one of claims 1 to 3, wherein the second nucleic acid strand is fully complementary to the first nucleic acid strand.

5. The interfering RNA molecule of any one of claims 1 to 3, wherein mismatches in the double-stranded region promote duplex unwinding.

6. The interfering RNA molecule of any one of claims 1 to 5, wherein the double-stranded region is about 20 to about 30 nucleotides in length.

7. The interfering RNA molecule of any one of claims 1 to 6, wherein the double-stranded region is about 25 nucleotides in length.

8. The interfering RNA molecule of any one of claims 1 to 3, wherein the first nucleic acid strand has at least 90% identity to SEQ ID NO: 3.

9. The interfering RNA molecule of claim 8, wherein the first nucleic acid strand comprises SEQ ID NO: 3.

10. The interfering RNA molecule of claim 8 or 9, wherein the second nucleic acid strand has at least 90% identity to SEQ ID NO: 4.

11. The interfering RNA molecule claim 10, wherein the second nucleic acid strand comprises SEQ ID NO: 4.

12. The interfering RNA molecule of any one of claims 1 to 3, wherein the first nucleic acid strand has at least 90% identity to SEQ ID NO: 5.

13. The interfering RNA molecule of claim 12, wherein the first nucleic acid strand comprises SEQ ID NO: 5.

14. The interfering RNA molecule claim 12 or 13, wherein the second nucleic acid strand has at least 90% identity to SEQ ID NO: 6.

15. The interfering RNA molecule of claim 14, wherein the second nucleic acid strand comprises SEQ ID NO: 6.

16. The interfering RNA molecule of any one of claims 1 to 3, wherein the first nucleic acid strand has at least 90% identity to SEQ ID NO: 7.

17. The interfering RNA molecule of claim 16, wherein the first nucleic acid strand comprises SEQ ID NO: 7.

18. The interfering RNA molecule of claim 16 or 17, wherein the second nucleic acid strand has at least 90% identity to SEQ ID NO: 8.

19. The interfering RNA molecule of claim 18, wherein the second nucleic acid strand comprises SEQ ID NO: 8.

20. The interfering RNA molecule of any one of claims 1 to 3, wherein the first nucleic acid strand has at least 90% identity to SEQ ID NO: 9.

21. The interfering RNA molecule of claim 20, wherein the first nucleic acid strand comprises SEQ ID NO: 9.

22. The interfering RNA molecule of claim 20 or 21, wherein the second nucleic acid strand has at least 90% identity to SEQ ID NO: 10.

23. The interfering RNA molecule of claim 22, wherein the second nucleic acid strand comprises SEQ ID NO: 10.

24. The interfering RNA molecule of any one of claims 1 to 23, wherein the interfering RNA molecule comprises a 3′ overhang in the first nucleic acid strand and/or the second nucleic acid strand.

25. The interfering RNA molecule of any one of claims 1 to 24, wherein one or more of the nucleotides in the double-stranded region comprise modified nucleotides.

26. The interfering RNA molecule of any one of claims 1 to 25, further comprising a carrier system.

27. A pharmaceutical composition comprising an interfering RNA molecule of any one of claims 1 to 26 and a pharmaceutically acceptable carrier.

28. A method for introducing an interfering RNA molecule that is capable of inhibiting expression of the R14del allele of the PLN gene in a cell, the method comprising:
contacting the cell with the interfering RNA molecule of any one of claims 1 to 26 or with the pharmaceutical composition of claim 27.

29. The method of claim 28, wherein the cell is a cardiac muscle cell.

30. The method of claim 29, wherein the cell is a cardiomyocyte.

31. The method of any one of claims 28 to 30, wherein the cell is in a subject.

32. The method of claim 31, wherein the subject is a carrier of the R14del allele of the PLN gene.

33. The method of claim 31 or 32, wherein the subject has been diagnosed with cardiomyopathy.

34. The method of any one of claims 31 to 33, wherein the subject is a human.

35. The method of any one of claims 28 to 34, wherein the interfering RNA molecule does not affect the contractility and/or the action potential of the cardiac muscle cell.

36. The method of any one of claims 28 to 34, wherein the interfering RNA molecule has a positive effect on the contractility and/or the action potential of the cardiac muscle cell.

37. A method for in vivo delivery of an interfering RNA molecule that is capable of inhibiting expression of the R14del allele of the PLN gene in a cell of a subject, the method comprising:
administering to the subject the interfering RNA molecule of any one of claims 1 to 26 or the pharmaceutical composition of claim 27.

38. The method of claim 37, wherein the cell is a cardiac muscle cell.

39. The method of claim 37 or 38, wherein the subject is a carrier of the R14del allele of the PLN gene.

40. The method of any one of claims 37 to 39, wherein the subject has been diagnosed with cardiomyopathy.

41. The method of any one of claims 37 to 40, wherein the subject is a human.

42. A method for treating cardiomyopathy in a subject carrying the R14del allele of the PLN gene, the method comprising:
administering to the subject a therapeutically effective amount of the interfering RNA molecule of any one of claims 1 to 26 or the pharmaceutical composition of claim 27.

43. A method for lowering the likelihood of at least one of malignant arrhythmias, sudden cardiac death, and/or a heart transplant in a subject carrying the R14del allele of the PLN gene, the method comprising:
administering to the subject a therapeutically effective amount of the interfering RNA molecule of any one of claims 1 to 26 or the pharmaceutical composition of claim 27.

44. The method of claim 42 or 43, wherein the subject has been diagnosed with cardiomyopathy.

45. The method of any one of claims 42 to 44, wherein the subject is a human.

46. The method of any one of claims 37 to 45, wherein the mode of administration is selected from the group consisting of oral, intranasal, intravenous, intraperitoneal. intramuscular, intraarticular, intralesional, subcutaneous, and intradermal.


SEQUENCE LISTING (13 sequences)

SEQ ID NO: 1 - RNA, 48 nt, synthetic construct
  source 1..48: mol_type = other RNA
actcgctcagctataagaagagcctcaaccattgaaatgcctcaacaa

SEQ ID NO: 2 - RNA, 45 nt, synthetic construct
  source 1..45: mol_type = other RNA
actcgctcagctataagagcctcaaccattgaaatgcctcaacaa

SEQ ID NO: 3 - RNA, 23 nt, synthetic construct
  source 1..23: mol_type = other RNA
ttcaatggttgaggctcttatag

SEQ ID NO: 4 - RNA, 23 nt, synthetic construct
  source 1..23: mol_type = other RNA
ctataagagcctcaaccattgaa

SEQ ID NO: 5 - RNA, 27 nt, synthetic construct
  source 1..27: mol_type = other RNA
  ncRNA 1..27: ncRNA_class = siRNA
atttcaatggttgaggctcttatagct

SEQ ID NO: 6 - RNA, 25 nt, synthetic construct
  source 1..25: mol_type = other RNA
  ncRNA 1..25: ncRNA_class = siRNA
ctataagagcctcaaccattgaaat

SEQ ID NO: 7 - RNA, 27 nt, synthetic construct
  source 1..27: mol_type = other RNA
  ncRNA 1..27: ncRNA_class = siRNA
tttcaatggttgaggctcttatagctg

SEQ ID NO: 8 - RNA, 25 nt, synthetic construct
  source 1..25: mol_type = other RNA
  ncRNA 1..25: ncRNA_class = siRNA
gctataagagcctcaaccattgaaa

SEQ ID NO: 9 - RNA, 27 nt, synthetic construct
  source 1..27: mol_type = other RNA
  ncRNA 1..27: ncRNA_class = siRNA
ttcaatggttgaggctcttatagctga

SEQ ID NO: 10 - RNA, 25 nt, synthetic construct
  source 1..25: mol_type = other RNA
  ncRNA 1..25: ncRNA_class = siRNA
agctataagagcctcaaccattgaa

SEQ ID NO: 11 - DNA, 78 bp, synthetic construct
  source 1..78: mol_type = other DNA
atggagaaagtccaatacctcacttgctcagctataagagcctcaaccattgaaatgcctcaacaagcacgtcaaaag

SEQ ID NO: 12 - DNA, 78 bp, synthetic construct
  source 1..78: mol_type = other DNA
tacctctttcaggttatggagtgaacgagtcgatattctcggagttggtaactttacggagttgttcgtgcagttttc

SEQ ID NO: 13 - protein, 26 aa, synthetic construct
MEKVQYLTCSAIRASTIEMPQQARQK